(19) United States
(12) Patent Application Publication
(10) Pub. No.: US 2022/0133330 A1
(43) Pub. Date: May 5, 2022

(54) THERAPEUTIC DEVICES FOR SEALING FLUID PASSAGEWAYS IN VENTILATORY AIRWAYS

(71) Applicant: Gyrus ACMI, Inc. d/b/a Olympus Surgical Technologies America, Westborough, MA (US)

(72) Inventors: Alyssa Philipps, Seattle, WA (US); Paul Taylor, Woodinville, WA (US)

(21) Appl. No.: 17/514,450

(22) Filed: Oct. 29, 2021

Related U.S. Application Data
(60) Provisional application No. 63/107,051, filed on Oct. 29, 2020.

(51) Int. Cl. A61B 17/12

(52) U.S. Cl. CPC A61B 17/12031; A61B 17/12131; A61B 17/12104

(57) ABSTRACT

A one-way lung valve for sealing fluid passageways in ventilatory airways. The valve includes a frame and a flexible membrane having respective opposed inner and outer surfaces and is supported by the frame. The flexible membrane is configured to transition between a compressed configuration and an occluding configuration in the ventilatory airways. The respective opposed inner and outer surfaces includes one or more features on the inner and/or outer surface to prevent the flexible membrane from forming one or more air gaps when the flexible membrane is in the occluding configuration position.

### PRIORITY CLAIM

This application claims the benefit of U.S. Provisional Application Ser. No. 63/107,051 filed Oct. 29, 2020, which is hereby incorporated by reference.

### DESCRIPTION OF THE RELATED ART

There is a continuing need for improved minimally invasive delivery of therapeutic devices to all portions of the respiratory system, particularly the lungs, bronchi and bronchiole, blood vessels, and lymphatic system. One such a therapeutic device is the one-way lung valve. The one-way lung valve, such as that produced by Spiration®, is placed in selected lung airways to occlude areas of the lung while still allowing mucus and trapped air to escape. Gaps may form in the membrane of the lung valve due do the airway compressing the valve.

### BRIEF SUMMARY OF EMBODIMENTS

Embodiments of the technology disclosed herein are directed to a one-way lung valve that redirects air away from diseased or damaged lung lobe to a healthier lung lobe, all while allowing trapped air and secretions to escape, so that patients may breathe easier. Unlike a stent, the unique design of the lung valve minimizes contact with the bronchial wall, maintains position to redirect air even in patients, and facilitates easy removal when needed. The valve includes a Nitinol frame covered with a polymer membrane and a plurality of anchors (e.g., five anchors) that securely engage the airway walls at the targeted treatment location. The procedure is considered minimally invasive and can be performed through a flexible bronchoscope. The benefits of the valve system treatment include, among others, a) Reduction in hyperinflation, b) Improvements in pulmonary function, c) Improved exercise tolerance, and d) Improved quality of life.

Accordingly, one aspect of the disclosed technology is directed to a therapeutic device for sealing fluid passageways in ventilatory airways. The therapeutic device includes a frame and a flexible membrane having respective opposed inner and outer surfaces and is supported by the frame. The flexible membrane is configured to transition between a compressed configuration and an occluding configuration in the ventilatory airways. The respective opposed inner and outer surfaces is constructed with having either one or more features on the inner surface or having one or more thicknesses formed on the outer surface to prevent the flexible membrane from forming one or more air gaps when the flexible membrane is in the occluding configuration but in a partially compressed configuration.

Another aspect of the disclosed technology is directed to a therapeutic device for sealing fluid passageways in ventilatory airways, which includes a plurality of struts, a central connector rod, and a plurality of anchor members, all of which are joined to one another at a central joint to define a frame. A flexible membrane is configured to be supported by the plurality of struts. The flexible membrane includes respective distal and proximal ends in which the frame extends in the proximal direction from the distal end. The flexible membrane includes a thicker region along the distal end as compared to the proximal end of the flexible membrane in which the thicker region forces the flexible membrane to completely seal the ventilatory airways without forming air gaps when therapeutic device is in an occluding configuration.

A further aspect of the disclosed technology is directed to therapeutic device for sealing fluid passageways in ventilatory airways. The therapeutic device includes a plurality of struts, a central connector rod, and a plurality of anchor members, all of which are joined to one another at a central joint to define a frame. A flexible membrane includes respective opposed inner and outer surfaces and is configured to be supported by the plurality of struts. The flexible membrane includes respective distal and proximal ends in which the frame extends in the proximal direction from the distal end. The inner surface includes one or more patterns defined by fishbone stiffening members, zigzag stiffening members, arcuate stiffening members, parallel stiffening members, or V-shape stiffening members formed thereto to prevent the flexible membrane from forming one or more air gaps when the flexible membrane is in the occluding configuration position.

A furthermore aspect of the disclosed technology is directed to a therapeutic device for sealing fluid passageways in ventilatory airways. Therapeutic device includes a frame and a flexible membrane supported by the frame. The flexible membrane includes one or more features that changes mechanical properties of the flexible membrane, thereby preventing the flexible membrane from forming one or more air gaps when the flexible membrane is in an occluding configuration position.

Other features and aspects of the disclosed technology will become apparent from the following detailed description, taken in conjunction with the accompanying drawings, which illustrate, by way of example, the features in accordance with embodiments of the disclosed technology. The summary is not intended to limit the scope of any inventions described herein, which are defined solely by the claims attached hereto.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1A is a sectional view of a prior art respiratory system having a prior art valve that seals fluid passageway in ventilatory airways.

FIG. 1B depicts a portion of FIG. 1 illustrating an enlarged view of the prior art valve.

FIG. 2A is a cross section of one of the prior art ventilatory airways having the valve being deployed therein and illustrating a prior art flexible membrane of the valve forming one or more air gaps when the flexible member is in the occluding configuration.

FIG. 2B is an end view of the prior art valve and one of the ventilatory airways depicted in FIG. 2A.

FIG. 3 is a perspective view of a therapeutic device in accordance with one embodiment of the technology described herein.

FIG. 4A is the sectional view of the respiratory system having the therapeutic device that seals fluid passageway in ventilatory airways in accordance with one embodiment of the technology described herein.

FIG. 4B is an end view of the therapeutic device and one of the ventilatory airways depicted in FIG. 4A illustrating the flexible membrane of the therapeutic device without air gaps when the flexible member is in the occluding configuration.

FIGS. 5A and 5B illustrate a comparison of folding pattern in the flexible membrane before and after the flexible member used one or more features that changes mechanical property of the flexible member.

FIGS. 6A-6L is a cross section of one of the ventilatory airways with the therapeutic device having various features formed onto the flexible member being deployed therein and illustrating a flexible membrane of the therapeutic device seals fluid passageway in ventilatory airways in accordance with an embodiment of the technology described herein when the flexible member is in the occluding configuration.

### DETAILED DESCRIPTION OF THE EMBODIMENTS

In the following description, various embodiments of the technology will be described. For purposes of explanation, specific configurations and details are set forth in order to provide a thorough understanding of the embodiments. However, it will also be apparent to one skilled in the art that the technology disclosed herein may be practiced without the specific details. Furthermore, well-known features may be omitted or simplified in order not to obscure the embodiment being described.

Before describing the disclosed system and/or technology in detail, it is useful to describe an example application with which the technology can be implemented. One such example application is in the field of bronchial valve treatment that represents the blocking technique of lung volume reduction modalities in which the lobe that meets certain clinically determined requirements is occluded by one or more one-way lung valves. These valves allow the air to exit during expiration but stop it from entering during inspiration. Therefore, a volume reduction in the occluded lobe, ideally a complete lobar atelectasis as the maximum result following valve therapy, can be achieved. The flexible membrane on the one-way valves may form air gaps that compromise complete sealing when the valve is compressed into smaller airways. The disclosed technology is capable of sealing across a diverse range of airways sizes and geometries. When compressed into smaller airways or non-circular geometries, the valve flexible membrane folds such that it seals without air gaps and as a result, this reduces the number of valve sizes required to cover a 5 mm range of airway sizes. Moreover, this simplifies the operator training and procedure time required to size an airway prior to placing a valve in ventilatory airways.

In one embodiment, the disclosed technology is directed to a therapeutic device such as one-way valve that is much less sensitive to airway size as compared to the other valves. One main factor that contributes to the sensitivity to airway size is the polyurethane-polycarbonate membrane of the valve that coats the proximal basket portion of the valve. The polyurethane-polycarbonate membrane has high tensile strength, but it lacks the softness and flexibility to conform to airways when compressed. Instead, the membrane may form air gaps that may reduce the performance of the valve. The disclosed technology uses the existing membrane material to seal air gaps completely and to increase the range over which each valve can perform the intended function.

In one embodiment, thicker membrane regions are formed at the distal end of the membrane and the membrane struts. The thicker membrane regions force the membrane to crease at longitudinally central locations of the membrane panel when the valve is compressed. This improves valve performance by reducing the air gaps that form when the valve is partially compressed. The flexible membrane of the valve is generally made of a biocompatible polyurethane or other thin, air impermeable material. Other derivative materials such as polyurethane-polycarbonate can be used as well.

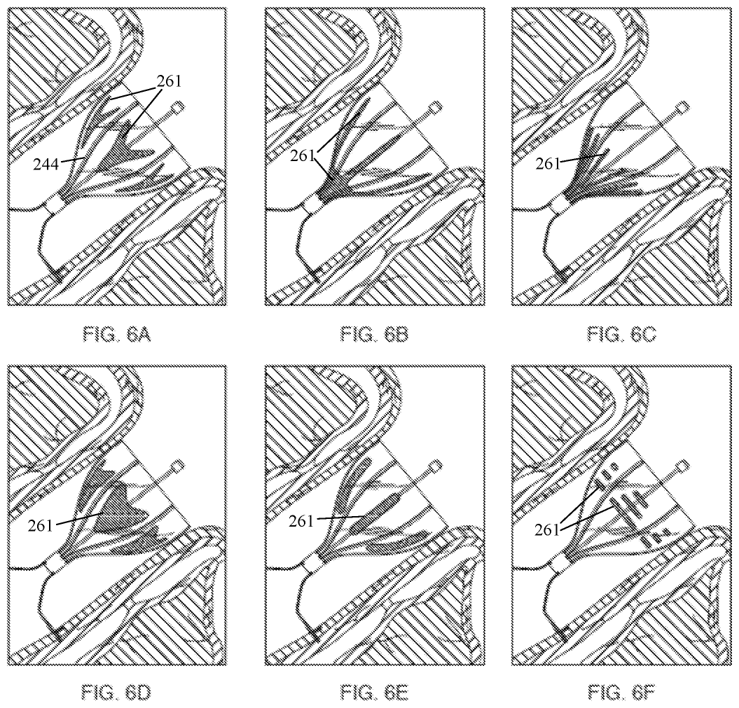

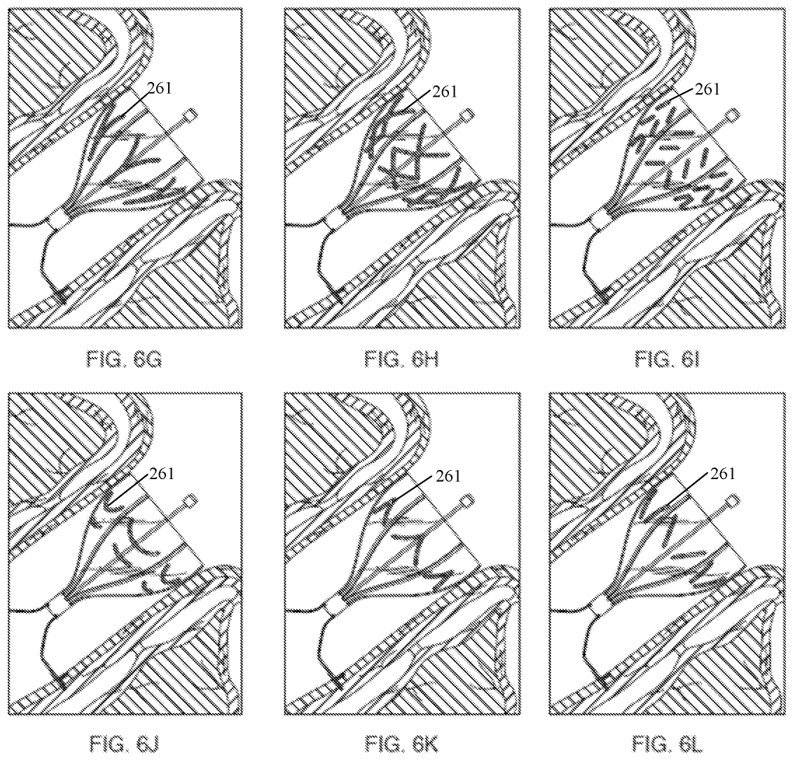

In another embodiment, the valve membrane includes stiffening members on an inner surface of the membrane. The stiffening members allow for the change in mechanical properties of the valve membrane without changing an outer surface of the valve membrane. The stiffening members alter the flex point(s) (i.e., redirect a flex force) of the membrane when the struts become slightly compressed so that the membrane folds at a location other than the proximal edge of the membrane. The stiffening members include micro-struts, such as those shown in FIGS. 6A-L, that can be fabricated to allow for precise folding or bending accordingly.

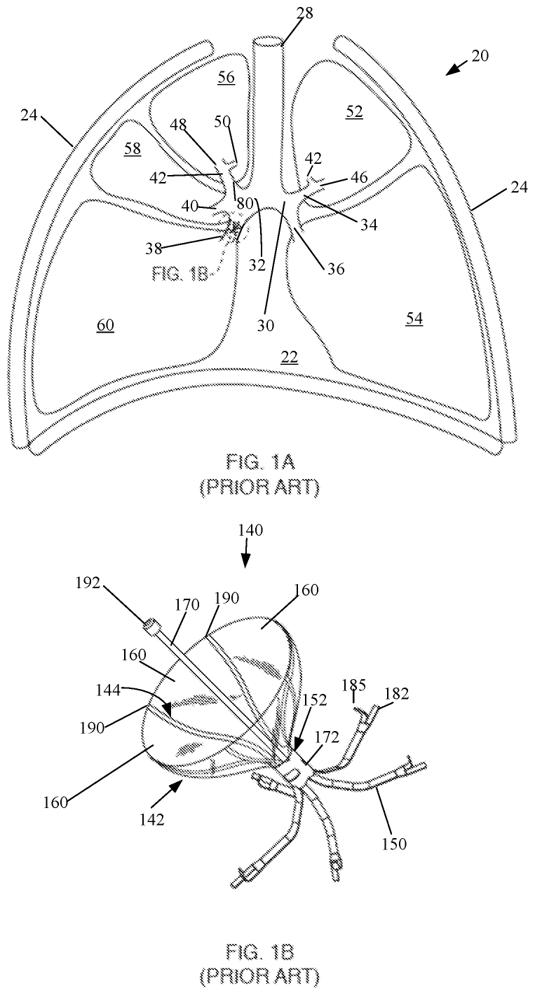

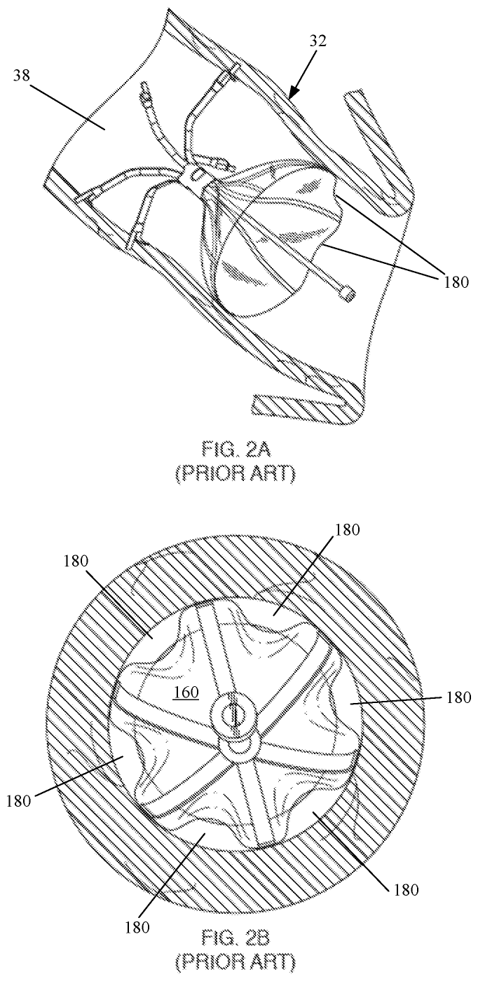

FIG. 1A is a sectional view of a prior art respiratory system 20 having a prior art valve or an occlusion device 140 (FIG. 1B) that seals fluid passageway 38 in ventilatory airways 30, 32 and FIG. 2B is an end view of the prior art valve 140 and one of the ventilatory airways 32 that is depicted in FIG. 2A. The respiratory system 20 resides within the thorax 28 that occupies a space defined by the chest wall 24 and the diaphragm 26. The detail description of the respiratory system 20 is provided in U.S. Pat. No. 6,929,637, the disclosure of which is incorporated in full herein by reference.

The respiratory system 20 includes trachea 28; left mainstem bronchus 30 and right mainstem bronchus 32 (primary, or first generation); and lobar bronchial branches 34, 36, 38, 40, and 42 (second generation). FIG. 1A also illustrates segmental branches 44, 46, 48, 49, and 50 (third generation). The respiratory system 20 further includes left lung lobes 52 and 54 and right lung lobes 56, 58, and 60. Each bronchial branch and sub-branch communicates with a different portion of a lung lobe, either the entire lung lobe or a portion thereof. As used herein, the term “fluid passageway” is meant to denote either a bronchi or bronchiole, and typically means a bronchial branch of any generation.

A characteristic of a healthy respiratory system is the arched or inwardly arcuate diaphragm 26. As the individual inhales, the diaphragm 26 straightens to increase the volume of the thorax 22. This causes a negative pressure within the thorax. The negative pressure within the thorax in turn causes the lung lobes to fill with air. When the individual exhales, the diaphragm returns to its original arched condition to decrease the volume of the thorax. The decreased volume of the thorax causes a positive pressure within the thorax, which in turn causes exhalation of the lung lobes.

Another characteristic of the respiratory system is the mucus flow from the lungs, or mucociliary transport system. Many pollution particles are inhaled as a person breathes, and the fluid or air passageways function as a very effective filter. The mucociliary transport system functions as a self-cleaning mechanism for all air or fluid passageways, including the lungs. The mucociliary transport system is a primary method for mucus clearance from distal portions of the lungs, and further constitutes a primary immune barrier for the lungs. The surface of air or fluid passageways is formed with respiratory epithelium (or epithelial membrane), which is covered with cilia and coated with mucus. As part of the mucociliary transport system, the mucus entraps many inhaled particles and moves them toward the larynx 28. The mucociliary transport system includes the metachronal ciliary beat of cilia on the respiratory epithelium that moves a continuous carpet of mucus and entrapped particles from the distal portions of the lungs past the larynx 28 and to the pharynx for expulsion from the respiratory system. The mucociliary transport system will also function as a self-clearing mechanism removing therapeutic agents placed in a lung portion and entrapped by the mucus. Additional description of the mucociliary transport system is provided in INTRA-BRONCHIAL OBSTRUCTING DEVICE THAT PERMITS MUCUS TRANSPORT filed May 9, 2002, application Ser. No. 10/143,353, which is owned by the Assignee, and which is incorporated herein by reference.

One embodiment of the occlusion device, illustrated in FIGS. 2A and 2B, includes an intra-bronchial valve (IBV) 140 for use in treating an air leak. The detail description of the intra-bronchial valve (IBV) 140 is provided in U.S. Pat. No. 9,622,752, the disclosure of which is incorporated in full herein by reference. The illustrated valve 140 is generally umbrella-shaped and includes a frame 142 having a plurality of struts 144 and a plurality of anchor members 150, all of which are joined together at a central joint 152. The struts 144 are generally configured to support a flexible membrane 160 on their outer surface 162. The intra-bronchial valve (IBV) 140 is also illustrated as including a central connector rod 170 adapted to be engaged in deploying and removing the intra-bronchial valve (IBV) 140.

As illustrated, the frame 142 may include a shape similar to an asymmetric hourglass, with one end provided with a flexible membrane and configured to act as a valve member, and the other end configured to anchor the valve against axial movement within an airway. According to one embodiment, the frame 142 (including the struts 144 and the anchors 150) can be made of a single tubular piece of a superelastic material such as Nickel-Titanium (also known as NiTi or NITINOL). The frame 142 can be machined, molded, or otherwise formed to create the desired functional elements as shown and described herein. In one embodiment, a hollow tube of NiTi is machined to form the struts 144 and anchors 150. The struts and anchors can be formed from the solid tube by making axial cuts along the tube to create the desired number of struts 144 and anchors 150 while leaving an uncut ring section 172 between the struts 144 and the anchors 150. The struts 144 and anchors 150 can then be bent into the desired shapes in such a way that the frame 142 will naturally assume the desired expanded shape at temperatures expected in an airway. Alternatively, the frame 142 can be made from sections of wire bent into the desired shapes. The skilled artisan will recognize that superelastic and/or “shape memory” materials such as NiTi typically require unique manufacturing processes involving substantial amounts of heat treating. The details of such manufacturing processes will be understood by those skilled in the art of Nickel-Titanium manufacturing.

The connector rod 170 typically includes a base concealed within the ring section 172 and is sized to be attached to the ring section 172 of the frame 142 at the central joint 152. The attachment of the connector rod 170 to the frame 142 can be accomplished by press fitting the base within the ring section 172 of the frame 142. If desired, the connection between the rod base and the ring section 172 can further include adhesives, welds, or any other suitable fastening means.

The frame 142 made of a superelastic material such as NiTi advantageously allows the valve to be compressed to occupy a very small axial shape during delivery, and to be released to assume a substantially larger shape when deployed. Additionally, a frame made of a shape-memory material will remain substantially elastic in its expanded and installed shape. Thus, the valve can be configured to have sufficient elasticity in its expanded shape to allow the intra-bronchial valve (IBV) 140 to expand and contract with the expansion and contraction of the bronchial walls 80, thereby maintaining the occlusion to distal airflow throughout repeated respiration cycles. Additionally, other intra-bronchial occlusion devices can be configured to expand and contract with the tissue of the bronchial walls.

In the illustrated embodiments, the frame 142 includes six struts 144 and five anchors 150. Other numbers of struts and/or anchors can alternatively be used. For example, the number of struts 144 and anchors 150 may be equal, and each individual strut may comprise a unitary structure with an individual anchor member.

The distal ends of the anchor members 150 include piercing tips 182 generally configured to puncture tissue of an air passageway wall 80 to retain the intra-bronchial valve (IBV) 140 in a desired location within the airway. As shown in FIG. 1B, the piercing tips 182 can include stops 185 configured to prevent the anchor members 150 from puncturing through the lung tissue beyond a desired depth.

During inspiration, a substantial air pressure differential can be built up with a high-pressure side on a proximal side of the valve. In the absence of any anchors, this pressure could potentially force the intra-bronchial valve (IBV) 140 distally within the airway. Therefore, it is desirable to anchor the intra-bronchial valve (IBV) against at least distal movement within the airway. In general, the fact that the intra-bronchial valve (IBV) 140 will allow expiratory airflow in a proximal direction past the device means that there will be a substantially smaller pressure differential across the valve during expiration (including coughing). Thus, the anchor arrangement are advantageously arranged to primarily prevent distal movement of the intra-bronchial valve (IBV) 140 within the airway. The intra-bronchial valve (IBV) 140 will also be anchored against proximal movement by the resilience of the anchor members 150 and the strut members 144 pressing against the bronchial wall 80. Also advantageously, when the connector rod 170 of the intra-bronchial valve (IBV) 140 is gripped and pulled proximally to remove the valve from an airway, the anchors 150 will collapse slightly, and the piercing tips 182 will release from engagement with the bronchial wall 80 as the valve is pulled proximally.

The shape of the struts 144 curve to further aid in allowing easy removal of the valve 140 from an airway by pulling the valve proximally. The end of struts 144 may be inwardly curved to prevent the struts 144 from snagging the tissue of the airway walls as the valve is drawn proximally. Suitable delivery and deployment devices are described in co-owned and co-pending U.S. patent application Ser. No. 10/052,875 filed Oct. 25, 2001 and Ser. No. 10/387,963 filed Mar. 12, 2003, both of which are incorporated by reference and made part of the instant disclosed technology.

The intra-bronchial valve (IBV) 140 is guided into a constricting funnel-shaped lumen to compress the IBV for loading into a delivery catheter as is described, for example in the Ser. No. 10/387,963 application mentioned above. As discussed in the '963 application, one embodiment of loading the valve 140 into a deliver catheter includes advancing the valve, rod-end first into a funnel-shaped constriction. The inwardly curved proximal strut ends will advantageously allow the valve to be smoothly advanced through such a funnel-shaped constriction without damaging the intra-bronchial valve (IBV).

As illustrated in FIG. 2A, the connector rod 170 extends from the central joint 152 proximally through the axial center of the valve 140. In one embodiment, the rod 170 is of such a length that it extends beyond the proximal ends 190 of the struts 144 when the valve 140 is in its expanded shape. The rod 170 can also be of such a length that the rod 170 and the struts 144 extend substantially the same distance from the central joint 152 when the valve 140 is in a fully compressed state (not shown). The connector rod 170 can be made of a biocompatible stainless steel, PVC, or any other suitably rigid biocompatible material as desired. The connector rod 170 is sufficiently rigid that when the proximal knob 192 of the rod 170 is gripped, the valve can be rigidly supported in a cantilevered manner. The connector rod 170 is also configured to facilitate removal of the IBV from an airway by gripping the proximal knob 192 of the rod 170 with standard or specially designed forceps and pulling proximally on the rod 170.

The flexible membrane 160 is generally made of a biocompatible polyurethane or other thin, air impermeable material. The membrane 160 can include tabs (not shown) which can be folded over the interior sides of the proximal ends 190 of the struts 144. Covering the proximal ends 190 of the struts 144 with tabs of membrane material advantageously prevents the struts 144 from digging into or snagging on the tissue of the bronchial wall, thereby aiding in removal of the IBV from an airway.

The flexible membrane 160 can be formed from a single, flat sheet of air impermeable material which can be sealed to the frame by adhesives, welds, heat seals or any other suitable manner to create an air-tight seal between a first (proximal) side and a second (distal) side of the membrane. Alternatively, the membrane 160 could be molded or thermoformed into a desired shape which can then be sealed to the frame 142.

Suitable occlusion devices can be provided in any size or configuration as desired. For example, in some embodiments, one-way valve occlusion devices having expanded outer diameters of between about 3 mm and about 9 mm can be used. Alternatively, valves having outer expanded diameters as small as 1 mm or less could be used.

As noted hereinbefore, suitable delivery and deployment devices can be used for delivering an intra-bronchial valve 140 to a target location in an airway of a lung, which include one of a number of known or newly developed devices. In one embodiment, means for delivery of an occlusion device to a target location includes a conventional bronchoscope (not shown) having a visualizing tip and at least one working lumen. A wide variety of bronchoscopes are commercially available, many of which will be suitable for carrying out portions of the air leak repair procedure described herein. Typical bronchoscopes have an outer diameter of about 5 mm, although larger or smaller bronchoscopes could also be used. Once the air leak has healed, a suitable means for removing an occlusion device can be introduced through the patient's airways to a position adjacent an occlusion device. Once in position, the means for removing an occlusion device can be operated in an appropriate manner to remove one or all of the occlusion devices. Alternatively, portions of the occlusion devices can be made from a substantially bioabsorbable polymer which can be substantially dissolved and absorbed by a patient's body fluids and tissue, thereby allowing bi-directional airflow to be resumed through a target location without the need for bronchoscopic removal of an occlusion device.

FIG. 2A is a cross section of one of the prior art ventilatory airways 38 having the valve 140 being deployed therein and illustrating a prior art flexible membrane 160 of the valve forming one or more air gaps 180 when the flexible member 160 is in the occluding configuration as seen best in FIG. 2B. As indicated previously, occasionally, the flexible membrane 160 on some of the valves form air gaps 180 that compromise complete sealing when the valve 140 is compressed into smaller airways. This sensitivity to airway size limits the range of airway sizes and geometries in which the valve can be operated.

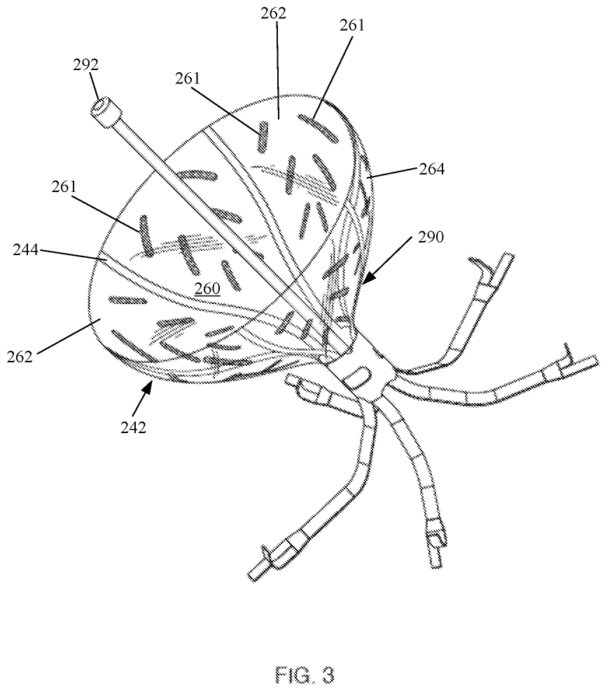

FIG. 3 is a perspective view of a therapeutic device 240 in accordance with an embodiment of the technology described herein. The difference between the valve 140 and the therapeutic device 240 depicted in FIG. 3 is that a flexible membrane 260 of includes various features 261 formed on an inner surface 262. The process of making and using the therapeutic device 240 in FIG. 3 is the same as the valve 140 described with respect to FIG. 1B and will not repeated to avoid redundancy. The flexible membrane 260 includes respective distal and proximal ends 290, 292 in which struts 244 of a frame extend in the proximal direction from the distal end 290. One example of the one or more struts 244 is defined by fishbone stiffening members formed on the inner surface 262 of the flexible membrane 260. The therapeutic device 240 is used for sealing fluid passageways 38 in ventilatory airways 32. The flexible membrane 260 is supported by the frame 242. The flexible membrane 242 includes one or more features 261 (i.e., micro-struts) that change mechanical properties of the flexible membrane 260, thereby preventing the flexible membrane 260 from forming one or more air gaps 180 when the flexible membrane 260 is in an occluding configuration.

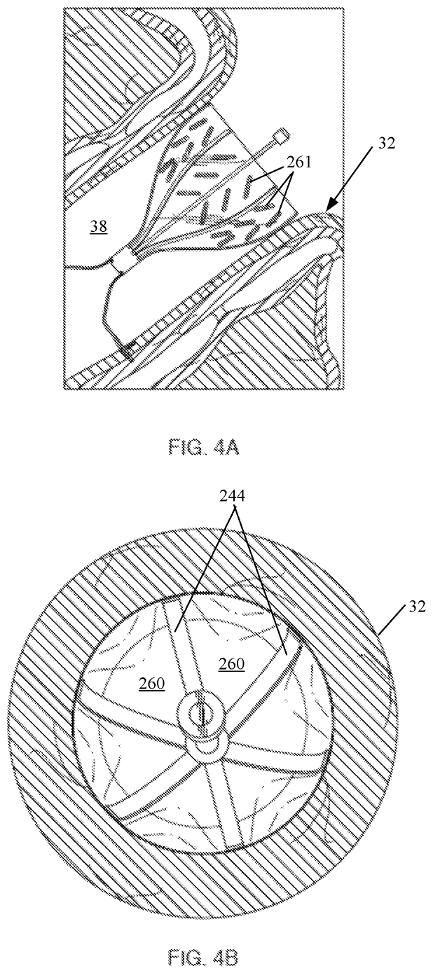

FIG. 4A is the sectional view of the respiratory system having the therapeutic device 240 that seals fluid passageway 38 in ventilatory airways in accordance with one embodiment of the technology described herein and FIG. 4B is an end view of the therapeutic device and one of the ventilatory airways depicted in FIG. 4A illustrating the flexible membrane 260 of the therapeutic device 240 without air gaps 180 when the flexible member 260 is in the occluding configuration. The therapeutic device 240 is a one-way valve used to prevent airflow to a lung lobe during n inhalation. The therapeutic device 240 or the one-way valve is capable of sealing across a diverse range of airways sizes and geometries. The therapeutic device 240 or the one-way valve significantly improves valve sealing in airways. When compressed into smaller airways or non-circular geometries, the valve flexible membrane 260 folds such that it seals without any air gaps or with limited air gaps as seen best in FIG. 4B and as a result, this improves valve efficiency and reduces the number of valve sizes required to cover, for example, a 5 mm range of airway sizes. Moreover, this simplifies the operator training and procedure time required to size an airway prior to placing a valve.

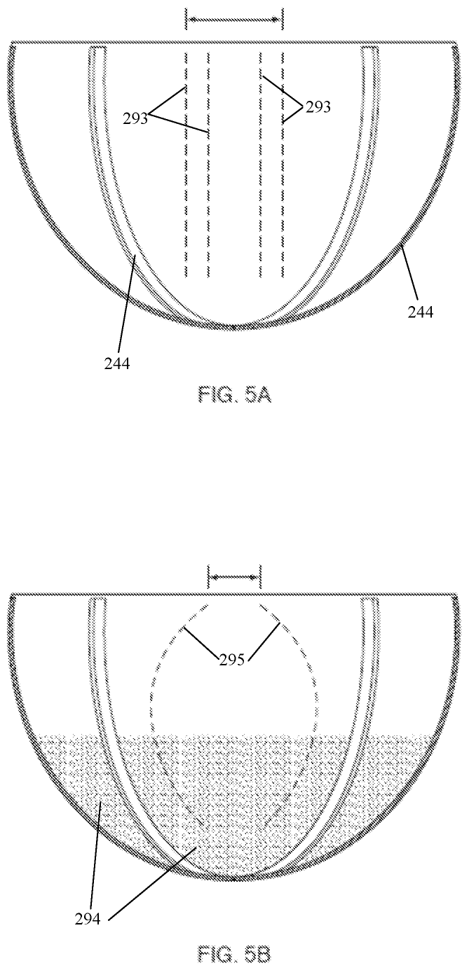

Referring now to FIGS. 5A and 5B, which illustrate a comparison of folding pattern in the flexible membrane 260 before and after the flexible member 260 used one or more features that changes mechanical property of the flexible member 260. For example, FIG. 5A illustrates the flexible membrane 260 that has a uniform thickness across the respective inner and outer surfaces 262 and 264 and thus, when the flexible membrane 260 is compressed under occluding configuration, the struts 244 are pushed inward and the membrane 260 having the uniform thickness vertically will naturally fold across the length of the membrane forming a channel A illustrated by the two pair of the parallel dash lines 293. On the other hand, FIG. 5B illustrates the flexible membrane 260 having a thicker region 294 along the distal end as compared to the proximal end of the flexible membrane. The thicker region 294 causes the flexible membrane to fold in an arcuate or diamond shape 295 as opposed to the parallel straight lines 293 shown in FIG. 5A and therefore, creates much better sealing for the valve 240 under occluding configuration. The thicker region 294 forces the flexible membrane 260 to crease in the center of the proximal end of the membrane when the valve is compressed.

The one or more features 261 are formed as fishbone stiffening members, zigzag stiffening members, arcuate stiffening members, parallel stiffening members, or V-shape stiffening members on the inner surface 262 of the flexible membrane as depicted in FIGS. 6A-6L. In one embodiment, one or more of the struts 244 can be constructed to have enhanced surface area to permit precise folding and/or bending of the frame without allowing the membrane to form any air gaps when the valve is in occluding configuration.

FIGS. 6A-6L is a cross section of one of the ventilatory airways with the therapeutic device 240 having various features formed onto the flexible membrane 260 being deployed therein and illustrating the flexible membrane 260 of the therapeutic device seals fluid passageway in ventilatory airways in accordance with an embodiment of the technology described herein when the flexible member is in the occluding configuration. One example of the various features includes fishbone stiffening members 261 depicted in respective FIGS. 3 and 61 that are formed on the inner surface 262 In one embodiment, as depicted in FIGS. 6A-6F, the struts 244 may include enhanced surface area 297 to permit precise folding and/or bending of the frame. The features 261 can be formed on the inner surface 262 by various means such as spraying or other techniques in the art. Suitable delivery and deployment devices can be used for delivering the therapeutic device 240 or the one-way valve to a target location in an airway of a lung, which include one of a number of known or newly developed devices.

In sum, the disclosed technology is directed to a therapeutic device for sealing fluid passageways in ventilatory airways. The therapeutic device comprises a frame and a flexible membrane having respective opposed inner and outer surfaces and is supported by the frame. The flexible membrane is configured to transition between a compressed configuration and an occluding configuration in the ventilatory airways. The respective opposed inner and outer surfaces is constructed with having either one or more features on the inner surface or having one or more thicknesses formed on the outer surface to prevent the flexible membrane from forming one or more air gaps when the flexible membrane is in the occluding configuration position.

The therapeutic device is a one-way valve used to prevent airflow to a diseased area of a lung during an inhalation. The flexible membrane includes respective distal and proximal ends in which the frame is extending in the proximal direction from the distal end. The flexible membrane includes a thicker region along the distal end as compared to the proximal end of the flexible membrane. The thicker region forces the flexible membrane to crease in the center of the proximal end of the membrane when the device is compressed. The flexible membrane seals the ventilatory airway without forming air gaps when in the occluding configuration position and substantially reduces the number valve sizes required to cover the ventilatory airway sizes. The therapeutic device is fabricated to reduce a procedure time required to size the ventilatory airway prior to placing the therapeutic device in the ventilatory airway. The one or more features are formed to change mechanical properties of the flexible membrane without changing the outer surface of the flexible membrane. The one or more features are defined by fishbone stiffening members, zigzag stiffening members, arcuate stiffening members, parallel stiffening members, or V-shape stiffening members on the inner surface of the flexible membrane. The flexible membrane is generally made of a biocompatible polyurethane or other thin, air impermeable material. The frame includes a plurality of struts, a central connector rod, and a plurality of anchor members, all of which are joined to one another at a central joint. The plurality of struts are configured to support the flexible membrane and each of the plurality of struts includes enhanced surface area to permit precise folding and/or bending of the frame.

The disclosed technology is further directed to a therapeutic device for sealing fluid passageways in ventilatory airways, which comprises a plurality of struts, a central connector rod, and a plurality of anchor members, all of which are joined to one another at a central joint to define a frame. A flexible membrane is configured to be supported by the plurality of struts. The flexible membrane includes respective distal and proximal ends in which the frame extends in the proximal direction from the distal end. The flexible membrane includes a thicker region along the distal end as compared to the proximal end of the flexible membrane in which the thicker region forces the flexible membrane to completely seal the ventilatory airways without forming air gaps when therapeutic device is in an occluding configuration.

The flexible membrane includes respective opposed inner and outer surfaces. The inner surface includes one or more patterns formed thereto to prevent the flexible membrane from forming one or more air gaps when the flexible membrane is in the occluding configuration position. The one or more patterns are defined by fishbone stiffening members, zigzag stiffening members, arcuate stiffening members, parallel stiffening members, or V-shape stiffening members on the inner surface of the flexible membrane. The flexible member is conical in shape with the outer surface positioned against the ventilatory airways, which allows trapped air and secretions to escape away from diseased or damaged lung.

The disclosed technology is also directed to therapeutic device for sealing fluid passageways in ventilatory airways. The therapeutic device includes a plurality of struts, a central connector rod, and a plurality of anchor members, all of which are joined to one another at a central joint to define a frame. A flexible membrane includes respective opposed inner and outer surfaces and is configured to be supported by the plurality of struts. The flexible membrane includes respective distal and proximal ends in which the frame extends in the proximal direction from the distal end. The inner surface includes one or more patterns defined by fishbone stiffening members, zigzag stiffening members, arcuate stiffening members, parallel stiffening members, or V-shape stiffening members formed thereto to prevent the flexible membrane from forming one or more air gaps when the flexible membrane is in the occluding configuration position.

The outer surface of the flexible member includes a thicker region along the distal end as compared to the proximal end of the flexible membrane in which the thicker region forces the flexible membrane completely seals the ventilatory airways. The one or more patterns are formed to change mechanical properties of the flexible membrane without changing the outer surface of the flexible membrane. The plurality of struts are configured to support the flexible membrane and each of the plurality of struts includes enhanced surface area to permit precise folding and/or bending of the frame.

Finally, the disclosed technology is directed to a therapeutic device for sealing fluid passageways in ventilatory airways. The therapeutic device comprises a frame and a flexible membrane supported by the frame. The flexible membrane includes one or more features that changes mechanical properties of the flexible membrane, thereby preventing the flexible membrane from forming one or more air gaps when the flexible membrane is in an occluding configuration position.

While various embodiments of the disclosed technology have been described above, it should be understood that they have been presented by way of example only, and not of limitation. Likewise, the various diagrams may depict an example construction or other configuration for the disclosed technology, which is done to aid in understanding the features and functionality that can be included in the disclosed technology. The disclosed technology is not restricted to the illustrated example construction or configurations, but the desired features can be implemented using a variety of alternative construction and configurations. Indeed, it will be apparent to one of skill in the art how alternative functional, logical or physical partitioning and configurations can be implemented to implement the desired features of the technology disclosed herein. Also, a multitude of different constituent parts names other than those depicted herein can be applied to the various parts. Additionally, with regard to flow diagrams, operational descriptions and method claims, the order in which the steps are presented herein shall not mandate that various embodiments be implemented to perform the recited functionality in the same order unless the context dictates otherwise.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent.

What is claimed is:

1. A device comprising;
a frame; and
a flexible membrane having respective opposed inner and outer surfaces and being supported by the frame, the flexible membrane configured to transition between a compressed configuration and an airway occluding configuration in the ventilatory airways, the respective opposed inner and outer surfaces comprise one or more features on the inner surface or on the outer surface to inhibit folding of a proximal end of the flexible membrane when in the airway occluding configuration.

2. The device of claim 1, wherein the device is a one-way valve used to prevent airflow into a targeted area of a lung.

3. The device of claim 1, wherein the flexible membrane includes a distal section and a proximal section.

4. The device of claim 3, wherein the flexible membrane is thicker at the distal section than at the proximal section.

5. The device of claim 4, wherein the thicker distal section forces the flexible membrane to crease in the center of the proximal end of the membrane when the device is at least partially compressed.

6. The device of claim 1, wherein the flexible membrane seals the ventilatory airway without forming air gaps when in the occluding configuration and substantially reduces the number valve sizes required to cover the ventilatory airway sizes.

7. The device of claim 1, wherein the one or more features are configured to change mechanical properties of the flexible membrane without changing the outer surface of the flexible membrane.

8. The device of claim 7, wherein the one or more features are defined by at least one of a fishbone stiffening member, a zigzag stiffening member, an arcuate stiffening member, a parallel stiffening member, or a V-shape stiffening member on the inner surface of the flexible membrane.

9. The device of claim 1, wherein the flexible membrane is generally made of a biocompatible polyurethane or other thin, air impermeable material.

10. The device of claim 1, wherein the frame comprises a plurality of struts, a central connector rod, and a plurality of anchor members, all of which are joined to one another at a central joint.

11. The device of claim 10, wherein the plurality of struts are configured to support the flexible membrane and wherein each of the plurality of struts includes enhanced surface area to permit precise folding and/or bending of the frame.

12. A therapeutic device for sealing fluid passageways in ventilatory airways comprising:
a plurality of struts, a central connector rod, and a plurality of anchor members, all of which are joined to one another at a central joint to define a frame; and
a flexible membrane configured to be supported by the plurality of struts, the flexible membrane includes respective distal and proximal ends in which the frame extends in the proximal direction from the distal end and wherein the flexible membrane includes a thicker region along the distal end as compared to the proximal end of the flexible membrane in which the thicker region forces the flexible membrane to completely seal the ventilatory airways without forming air gaps when therapeutic device is in an occluding configuration.

13. The therapeutic device of claim 12, wherein the flexible membrane includes respective opposed inner and outer surfaces wherein the inner surface includes one or more patterns of thicker membrane material configured to prevent the flexible membrane from forming one or more air gaps when the flexible membrane is in the occluding configuration.

14. The therapeutic device of claim 13, wherein the one or more patterns comprise at least one of a fishbone pattern, a zigzag pattern, an arcuate pattern, a parallel pattern, or a V-shape pattern on the inner surface of the flexible membrane.

15. The therapeutic device of claim 14, wherein the flexible member is conical in shape with the outer surface positioned against the ventilatory airways, which allows trapped air and secretions to escape away from diseased or damaged lung.

16. A therapeutic device for sealing fluid passageways in ventilatory airways comprising:
a plurality of struts, a central connector rod, and a plurality of anchor members, all of which are joined to one another at a central joint to define a frame; and
a flexible membrane having respective opposed inner and outer surfaces and being configured to be supported by the plurality of struts, the flexible membrane includes respective distal and proximal ends in which the frame being extending in the proximal direction from the distal end and wherein the inner surface includes one or more patterns defined by fishbone stiffening members, zigzag stiffening members, arcuate stiffening members, parallel stiffening members, or V-shape stiffening members formed thereto to prevent the flexible membrane from forming one or more air gaps when the flexible membrane is in the occluding configuration.

17. The therapeutic device of claim 16, wherein the outer surface includes a thicker region along the distal end as compared to the proximal end of the flexible membrane in which the thicker region forces the flexible membrane completely seals the ventilatory airways.

18. The therapeutic device of claim 16, wherein the one or more patterns are formed to change mechanical properties of the flexible membrane without changing the outer surface of the flexible membrane.

19. The therapeutic device of claim 16, wherein the plurality of struts are configured to support the flexible membrane and wherein each of the plurality of struts includes enhanced surface area to permit precise folding and/or bending of the frame.
